(19) United States
(12) Patent Application Publication
(10) Pub. No.: US 2017/0224870 A1
(43) Pub. Date: Aug. 10, 2017

(54) METHOD OF PACKAGING AMNIOTIC MEMBRANE

(71) Applicants: Darío Hernán Vasquez Zuloaga, Santiago (CL); Cristhian Alejandro Urzua Salinas, Santiago (CL)

(72) Inventors: Darío Hernán Vasquez Zuloaga, Santiago (CL); Cristhian Alejandro Urzua Salinas, Santiago (CL)

(21) Appl. No.: 15/502,006

(22) PCT Filed: Aug. 5, 2015
(86) PCT No.: PCT/CL2015/000039

§ 371 Date: Feb. 6, 2017

(30) Foreign Application Priority Data
Aug. 6, 2014 (CL) 2088-2014

(51) Int. Cl. A61L 27/36; B65B 55/16; C12N 5/00; A61L 2/00; A61K 35/50; A61L 27/54

(52) U.S. Cl. CPC A61L 27/3604; A61K 35/50; A61L 27/3691; A61L 27/54; C12N 5/0068; A61L 2/0047; B65B 55/16; A61F 2/10

(57) ABSTRACT

A method of packaging amniotic membrane that maintains its structure to be used as a graft includes attaching the amniotic membrane to a support, dimensioning the amniotic membrane together with the support through an inert cutting device or guillotine, gluing the amniotic membrane together with the support to the interior of a container, and sterilizing the amniotic membrane. A packaged amniotic membrane includes a dimensioned sterilized amniotic membrane attached to a support, wherein the amniotic membrane is glued together with the support to a rigid, sterilized container.

### TECHNICAL FIELD

This disclosure relates to a method of packaging human amniotic membrane (AM) to obtain a product that is ready to be used as a graft in surgeries, as a support for culturing or for diagnostic or therapeutic purposes. The method or procedure keeps the structure of the amniotic membrane, for example, to be used as a graft in surgeries such as ocular surgery.

### BACKGROUND

The transplant of preserved human amniotic membrane can be considered one of the biggest developments related to surgery of the surface of the eye or skin. The first documented ophthalmic use of AM in the international literature was almost 70 years ago. However, transplant of the amniotic membrane has been done with large numbers of patients only since 1995, and with very good results (V. Jhanji, A. L. Young, J. S. Mehta, N. Sharma, T. Agarwal and R. B. Vajpayee, Management of corneal perforation, Sury Ophthalmol, 2011, 56(6): p. 522-538).

Several diseases of the ocular surface still represent a clinical challenge in ocular surgery. Within that group are included persistent epithelial defects of the cornea, acute chemical or thermal burns with long term loss of integrity of the superficial epithelium, and diseases of the mucus membranes that produce scarring of the conjunctiva as a result of inflammation. Since introduction of modern preservation methods, the inner most lining of the placenta, AM, procured under sterile conditions after a caesarean section, has experienced a renaissance as a substitute for the basal membrane. For example, during 2008, in Germany a total of 2,308 transplants of the amniotic membrane were done. The possible applications of AM go from a graft to heal wounds, as a support for cellular culture, as a patch, to being utilized as a substrate for the ex-vivo growth of the epithelium of the ocular surface, for the growth of microorganisms with diagnostic, therapeutic or industrial purposes. Each type of application leads to different histological patterns of integration of the AM with the recipient tissue. Indications for the use of AM in reconstructive surgery of the ocular surface are, for example, persistent defects of the corneal epithelium, corneal ulceration of different etiology, covering of defects after surgical removal of extensive defects of the conjunctiva such as, for example, tumors, acute chemical burns, symblepharon and reconstruction of the fornix in diseases related to scarring of the conjunctiva and corneal limbal stem cell deficiency with simultaneous grafting of stem cells (D. Meller, M. Pauklin, H. Thomasen, H. Westekemper and K. P. Steuhl, Amniotic membrane transplantation in the human eye, Dtsch Arztebl Int, 2011, 108(14): p. 243-248).

### SUMMARY

We provide a method of packaging amniotic membrane that maintains its structure to be used as a draft including attaching the amniotic membrane to a support, dimensioning the amniotic membrane together with the support through an inert cutting device or guillotine, gluing the amniotic membrane together with the support to the interior of a container, and sterilizing the amniotic membrane.

We also provide a packaged amniotic membrane including a dimensioned sterilized amniotic membrane attached to a support, wherein the amniotic membrane is glued together with the support to a rigid, sterilized container.

We further provide a patch including a support for culturing or diagnostic purposes including the packaged amniotic membrane including a dimensioned sterilized amniotic membrane attached to a support, wherein the amniotic membrane is glued together with the support to a rigid, sterilized container.

We also further provide a patch including a device for release of growth factors including the packaged amniotic membrane including a dimensioned sterilized amniotic membrane attached to a support, wherein the amniotic membrane is glued together with the support to a rigid, sterilized container.

### DETAILED DESCRIPTION

The AM forms part of the fetal annexes that provide oxygen and nutrients to the fetus during gestation. It is composed of a monostratified epithelium of cubic cells suspended by a thick basal membrane and connective tissue.

Unique characteristics of the AM that can be highlighted are:
Reduction of inflammation: The AM has the capability of inhibiting immune responses due to the high concentration of immunomodulatory cytokines such as transforming growth factor beta;
Antimicrobial properties;
Promotion of reepithelialization; and
Reduced immunogenicity: The AM has a low rate of rejection determined by a low expression of molecules of the major histocompatibility complex (MEW).

This group of properties has been fundamental for the beneficial use of AM as a graft in pathologies of the surface of the eye or the skin in which the inflammatory phenomena constitutes a common final pathway that determines structural and functional damage.

Until today, the use of AM has been described for the treatment of different types of ophthalmic pathologies. The processing has included the use of gamma radiation to ensure sterile conditions. Within the ocular diseases that affect human beings all around the world, there is the challenge of reconstructing the ocular surface due to the morphological and functional complexity of the ocular structure. A key issue for resolution of ocular or skin pathologies is the need for a biocompatible matrix that can integrate itself and maintain the distinctive characteristics in which different types of tissues (epithelial, connective, vascular, glandular) and cells (stem cells, immune cells, mature cells) combine.

The use of fresh AM presents a high risk of transmission of infectious and contagious diseases because of the possibility of the donor being in the “window period” of a particular disease, that means, the period of time in which the diagnostic tests are negative even though the donor is a carrier and, as such, a potential transmitter of the disease.

The possibility of processing and preserving the transplantable organs and tissues during different periods of time allows for the performance of diagnostic tests for infections and contagious diseases outside of this window period, significantly reducing the probability of transmission of infections from the donor to the recipient (L. Buzzonetti, G. Petrocelli and P. Valente, Amniotic membrane transplantation in corneal melting after anterior lamellar kerato-plasty assisted by femtosecond laser in children, Eur J Ophthalmol, 2012, 22(3): p. 477-480).

On the other hand, this favors generation of a process for distribution to places distant from the production site, decentralizing the implantation procedure, as occurs with the transplanting of solid organs such as the heart and kidneys.

In Chile, until today, there are no patented methods or procedures to process amniotic membrane. Application of gamma radiation to the fresh and processed forms of the AM has been described as a factor that alters the biomechanical properties of the AM.

The processed and packaged AM can be distributed to any medical center in Chile or in other countries, if and only if a series of preservation conditions are maintained (cold chain).

There exists a current problem in generation of good quality AM that can be safely distributed as a product or as medical supplies, and in a way that allows the graft to get to the place of the surgery in optimal condition for use.

U.S. Pat. No. 6,152,142 describes the use of an amniotic membrane for ocular surgery. However, it does not describe the manufacturing process. Even though it does not describe the packaging method, when using such graft it can be noticed that, to preserve it, physio-chemical modifications have been made, altering the intrinsic properties of the amniotic membrane. Thus, the resulting graft presents lower flexibility, lower quality and difficulty in surgical manipulation.

On the other hand, it should be highlighted that the process of obtaining and packaging the graft presents a series of technical difficulties:
After the washing process, the amniotic membrane is very hard to manage due to its elastic-adhesive consistency and transparency.

To address the problem of difficult handling, the AM has been attached to a support to make the AM manageable to be able to extend it and make cuts with defined dimensions that keep the epithelial surface identifiable.

The support that the AM is attached to should fulfill a series of conditions, first it should be capable of attaching the AM to its surface, and be an inert material that does not interact nor react with the AM. Additionally, the support material should be capable of reducing its affinity for the AM at the moment of hydration of the AM to proceed with detachment of the AM from the support.

We provide a method of packaging an amniotic membrane that maintains its structure to be used as a graft in surgery, as support for culture, diagnostic or therapeutic purposes. The method of packaging the amniotic membrane comprises adhering the amniotic membrane to a support, dimensioning the amniotic membrane together with the support using an inert cutting device or guillotine, gluing the amniotic membrane together with the support to the interior of a container and sterilizing the amniotic membrane. The amniotic membrane is sterilized with UV radiation.

The amniotic membrane can be sterilized in advance with UV radiation, and then a sterile support of ethylene-vinyl acetate (EVA) or expanded polystyrene (plumavit) is attached. The stage where the amniotic membrane is attached to the sterile support is performed through dehydration of the amniotic membrane under a laminar flow cabinet.

The amniotic membrane together with the support are glued to the interior of a rigid container, where the rigid container is made of material such as glass, polystyrene, plumavit, plastic or acrylic, that is resistant to mechanical trauma and protects the membrane integrity.

The amniotic membrane together with the support is attached to the interior of a rigid container through synthetic adhesives such as cyanoacrylates or other glues.

Dimensioning of the amniotic membrane together with the support is performed with a double blade guillotine or shears made of a rigid, sharp and sterilizable material such as stainless steel.

We also provide a packaged amniotic membrane composed of a dimensioned, sterilized amniotic membrane attached to a support, where the amniotic membrane is glued together with the support to a rigid, sterilized container. The amniotic membrane can be dimensioned in patches of 1 cm. The amniotic membrane can be dimensioned in patches of 5 cm comprising one or more layers. The amniotic membrane can be enriched with proteins or plasma factors. Also, the amniotic membrane can be enriched with proteins or plasma factors from the same patient.

The packaged amniotic membrane serves as a support for culture or with diagnostic purposes, as a device for the release of growth factors.

With our procedures, the morphological and functional characteristics of the human amniotic membrane can be preserved so the graft can get to the place of the surgery in optimal condition for use.

The processed amniotic membrane is ready to be used in any kind of wound or surgery of the surface of the body such as ulcers of the cornea or other eye and skin surfaces, surgeries of the conjunctiva, diabetic type skin ulcers, varicose, or others, reconstructive surgeries, healing of wounds in any area, mouth lesions, dental surgeries, oropharyngeal repair, ear surgery, ear, eardrums, repair of internal structures such as solid or hollow organs, joints, ligaments or other membranes. Likewise, the amniotic membrane can be later processed to obtain a membrane concentrate, or pulverization, lyophilization, fragmentation or purification of all of it or of any of its components. The membrane can also be used as a patch to cure wounds or as a surgical graft for animals, on the surface as well as in the interior of their bodies. Additionally, the membrane can be used as a support for cellular culture, growth of other microorganisms with diagnostic, therapeutic or industrial purposes, as well as for experimentation purposes. The membrane processed in this way can also be used as a vehicle for the delivery of pharmaceuticals, active compounds, antibodies, natural extracts or other different types of chemical compounds.

A support of ethylene-vinyl acetate (EVA) or plumavit (expanded polystyrene) was used. This support is used after being previously sterilized and complies with all the previously mentioned conditions. The EVA is a thermo-plastic polymer formed by repetitive units of ethylene and vinyl acetate. It is characterized by being easy to maneuver, easy to adhere, sterilizable, elastic, non-toxic, easy to cut and having low water absorption. This allows a double function, first that the dehydrated membrane adheres to its surface, and that after being rehydrated before its use, the EVA loses affinity and adherence to the AM allowing the AM to be removed integrally without traction and without suffering any damage. Also, due to its elasticity, the EVA can absorb any vibration or mechanical trauma that could possibly damage the membrane.

Once the AM is attached to the support it can be extended, dehydrated, irradiated, cut and easily manipulated.

A support of expanded polystyrene or paper can also be used.

EVA, plumavit (expanded polystyrene), paper or even nitrocellulose can be used as support materials. Preferably, EVA is utilized as the support material.

The EVA support presents characteristics that are superior to the nitrocellulose support previously described in the state of the art because the nitrocellulose support absorbs large quantities of water and is rigid, which makes detachment of the amniotic membrane more difficult at the time of use and limits the characteristic flexibility of this tissue.

The use of the amniotic membrane in patients requires a correct and precise dimensioning of the graft. The cut of the AM can be done with scissors or prefabricated metallic matrices. This presents the difficulty of not being operator dependent, and of generating irregular cuts in the AM due to the traction generated by cutting with scissors.

To solve this problem, an inert cutting device or shear was used such as a cut with a metallic blade guillotine. The cutting device should be made of inert material, not reactive such as stainless steel, previously sterilized, to make precise and clean cuts in the amniotic membrane without generating traction or folding the membrane. Cutting of the amniotic membrane previously attached to the support using a sterile guillotine with a stainless steel blade should be done inside a laminar flow cabinet under sterile conditions. In this way, the amniotic membrane can be dimensioned correctly without altering its quality.

When the amniotic membrane dries, it breaks easily. When cutting the amniotic membrane with a single blade guillotine, the cut amniotic membrane tends to break or crack. To avoid this problem of cracking, the cut is made with a double blade guillotine or shears. Even when the cutting of the amniotic membrane is made under wet conditions, the resulting cut looks better if a double blade guillotine or shear is used.

The amniotic membrane is a tissue that can be damaged during storage and transport from the laboratory to the surgery room, therefore, it should be protected by a container or rigid structure that can maintain its integrity. Additionally, it is necessary to have a container that can contain the water or saline solution for the hydration necessary for the graft to detach from the support. This problem has been resolved through the attachment of a support, together with the amniotic membrane, to the interior of a rigid sterile container whose material is resistant to mechanical trauma and protects the integrity of the membrane. In this way, the sterile, rigid container has an inferior part and a superior lid protects the graft and, at the same time, as a container to hydrate the membrane, since water or saline solution can be added directly inside the inferior part of the container that works, at the same time, as a receptacle. The support can be made of EVA or plumavit.

Adhesion of the AM together with the support to the interior of a rigid container should be done with a type of adhesive that is not reactive and that has been previously demonstrated not to damage biological tissues to maintain the quality of the product.

Glues from the cyanoacrylate family are used due to the fact that they are adhesives approved for use in medicine. The cyanoacrylate is an acrylic resin that polymerizes rapidly in the presence of water forming long and strong chains.

The family of cyanoacrylate compounds is widely used as tissue adhesives (of living biological tissues) to replace the surgical suture for wound closure such as sealants and hemostatics. In this way, based on the biocompatibility of the adhesives of the cyanoacrylates family of compounds, the risk of damage to the amniotic membrane can be avoided.

We provide a method of packaging of human amniotic membrane (AM) to obtain a product ready to be used as a graft in surgery that is sterile and that preserves the biological and biomechanical properties of the tissue.

The packaging method for the AM is framed within a group of sequential steps:
1. Obtain a placenta from an elective caesarean section procedure. The mother is informed of the procedure and informed consent is obtained from her.
2. Separate the AM from the rest of the fetal annexes (chorion).
3. Wash the AM with solutions that contain antibiotics and antifungals, eliminating leftover blood.
4. PROCEDURE TO PACKAGE THE AMNIOTIC MEMBRANE:
   A. Attachment by dehydration of the AM to a support of: sterile ethylene-vinyl acetate, plumavit or paper:
   B. Cut with a sterilized stainless steel guillotine;
   C. Gluing with synthetic adhesives (cyanoacrylate or other glues) the amniotic membrane together with the support to the interior of a rigid container made of: glass, polystyrene, plumavit, plastic or acrylic; and
   D. Irradiation with UV light for a period of 20 to 60 minutes.
5. Protection of the container in the interior of a sterile trilaminar envelope (polystyrene-polyamide-aluminum) thermo-sealed for storage and transport.

The amniotic membrane can be dimensioned in squares of 1 cm by 1 cm for ocular applications, or it can be dimensioned in squares of 5 cm by 5 cm for skin applications, for example, skin patches. The amniotic membrane is dimensioned in one or more layers. The amniotic membrane can also be enriched with growth factors or other types of pharmaceuticals or molecules.

### EXAMPLE

The AM obtained with our procedure has been evaluated from a morphological and functional point of view.

As an initial evaluation, the AM was tested in a standard culture and for fungus without observing the presence of microorganisms.

To evaluate the morphological characteristics, the AM was stained with Hematoxylin-Eosin, there was observed by optical microscopy a morphology similar to the one described in the literature, meaning, an epithelium with a single layer of cuboidal cells with a basal membrane and subjacent connective tissue.

Later, the functionality of the AM was explored, bearing in mind that the principal goal for the use of the AM in ophthalmology was the necessity for a matrix that allows for reconstruction of the ocular surface.

In this sense, the processed AM was evaluated as a matrix for the growth of epithelial cells. A Human Retinal Pigment Epithelial Cell Line was used (ARPE-19). These cells were plated (seeded) over the processed AM and adhesion and growth of the ARPE-19 was observed.

The amniotic membrane processed through this packaging method has been used and grafted in 20 Chilean patients with a 95% surgical success rate, defined as an anatomic improvement of the ocular surface and an improvement of the visual acuity. The pathologies treated include: pterygium, tumors of the conjunctiva, caustic burns, symblepharon and glaucoma.

This graft has been used in humans, but it has other potential applications in animals.

The amniotic membrane can also be used enriched with growth factors for which the amniotic membrane is submerged in blood, serum or plasma rich in proteins and growth factor. The amniotic membrane can be enriched with added growth factors for which the amniotic membrane is submerged in blood, serum or plasma rich in proteins and externally added growth factors. The amniotic membrane enriched in this way can be applied to the skin or to any body surface, including ocular tissue.

The amniotic membrane can also be enriched by submerging it in serum or plasma rich in platelets coming from the same patient. The serum or plasma rich in platelets from the same patient can also be enriched with growth factors to obtain an enriched amniotic membrane, or else as a device for the sustained release of different types of pharmaceuticals.

1-14. (canceled)

15. A method of packaging amniotic membrane that maintains its structure to be used as a graft comprising attaching the amniotic membrane to a support, dimensioning the amniotic membrane together with the support through an inert cutting device or guillotine, gluing the amniotic membrane together with the support to the interior of a container, and sterilizing the amniotic membrane.

16. The method according to claim 15, wherein the amniotic membrane is sterilized by UV radiation.

17. The method according to claim 15, wherein the amniotic membrane is previously sterilized with UV radiation and then attached to a sterilized support made of ethylene-vinyl acetate or expanded polystyrene.

18. The method according to claim 15, wherein attaching the amniotic membrane to a sterilized support is performed by dehydration of the amniotic membrane under a laminar flow cabinet.

19. The method according to claim 15, wherein the amniotic membrane together with the support is glued to the interior of a rigid container, where the rigid container is made of a material comprising glass, polystyrene, plumavit, plastic or acrylic, that is resistant to mechanical trauma and protects integrity of the membrane.

20. The method according to claim 15, wherein the amniotic membrane together with the support is glued to the interior of a rigid container through synthetic adhesives comprising cyanoacrylates or other glues.

21. The method according to claim 15, wherein the dimensioning of the amniotic membrane together with the support is performed with a double blade guillotine or shear made of rigid, sharp and sterilizable material.

22. A packaged amniotic membrane comprising a dimensioned sterilized amniotic membrane attached to a support, where the amniotic membrane is glued together with the support to a rigid, sterilized container.

23. The packaged amniotic membrane according to claim 22, wherein the amniotic membrane is dimensioned in patches of 1 cm.

24. The packaged amniotic membrane according to claim 22, wherein the amniotic membrane is dimensioned in patches of 5 cm and comprises one or more layers.

25. The packaged amniotic membrane according to claim 22, wherein the amniotic membrane is enriched with proteins or plasma factors.

26. The packaged amniotic membrane according to claim 22, wherein the amniotic membrane is enriched with proteins or plasma factors from the same patient.

27. A patch comprising a support for culturing or diagnostic purposes comprising the packaged amniotic membrane according to claim 22.

28. A patch comprising a device for release of growth factors comprising the packaged amniotic membrane according to claim 22.
